(19) United States
(12) Patent Application Publication
(10) Pub. No.: US 2026/0035687 A1
(43) Pub. Date: Feb. 5, 2026

(54) SAMPLING DEVICE

(71) Applicant: UNIVERSAL BIO RESEARCH CO., LTD., Chiba (JP)

(72) Inventor: Hideji TAJIMA, Chiba (JP)

(21) Appl. No.: 18/997,970

(22) PCT Filed: Jul. 24, 2023
(86) PCT No.: PCT/JP2023/026995

§ 371 Date: Jan. 23, 2025

(30) Foreign Application Priority Data
Jul. 26, 2022 (JP) 2022-118439

(51) Int. Cl. C12N 15/10; G01N 35/10

(52) U.S. Cl. CPC C12N 15/1013; G01N 35/1011; G01N 2035/1013; G01N 2035/1053

(57) ABSTRACT

A sampling device 1 includes: an electrode nozzle 2, a nozzle moving mechanism moving the electrode nozzle 2 in a vertical direction, a dispensing tip 3 attached to the electrode nozzle 2, a positive electrode 5a and a negative electrode 5b attached to the electrode nozzle 2 and extending along the dispensing tip 3, a current detector 7 detecting a value of a current flowing between the positive electrode 5a and the negative electrode 5b, a sample container housing a sample solution, and a controller controlling the nozzle moving mechanism. The controller drives the nozzle moving mechanism to move the electrode nozzle 2, specifies a position of a predetermined component layer from variation of the value of the current, and the dispensing tip 3 in the vertical direction, and drives the electrode nozzle 2 to sample the predetermined component layer.

### TECHNICAL FIELD

The present invention relates to a sampling device sampling a predetermined component layer from a sample such as centrifuged whole blood.

### BACKGROUND ART

When whole blood housed in a sample container is centrifuged at about 1500 rpm, the whole blood is separated into a plurality of component layers. During centrifugation, the whole blood is diluted, and an anticoagulant (such as sodium citrate) is added thereto.

As illustrated in FIG. 6, the plurality of component layers are an upper layer A, a centrifugal agent layer B containing the anticoagulant and the like, and a red blood cell layer C in order from the top inside a sample container 10. The upper layer A includes a blood serum/blood plasma layer A1 on an upper side and a buffy coat layer A2 on a lower side. The blood serum/blood plasma layer A1 is a mixed solution layer containing cancer cells, exosomes, proteins, and the like. The buffy coat layer A2 is an immune cell solution layer.

Patent Literature 1 describes a blood component separation device separating centrifuged blood components. It is described that the blood component separation device presses a master bag housing centrifuged whole blood, detects interfaces of components sent from an upper part thereof, and sends the components to respective different slave bags. It is described that the interfaces are detected by an optical sensor.

### CITATION LIST

### Patent Literature

Patent Document 1: Japanese Patent Laid-Open No. 2003-135591

### SUMMARY OF INVENTION

### Technical Problem

The blood component separation device in Cited Document 1 forcibly sends the blood components by pressing the container such as the master bag. Therefore, the interfaces of the plurality of blood component layers are likely to be nonuniform, and the plurality of blood component layers may not be appropriately separated. Further, the blood component layers are separated in order from an upper layer. Therefore, it may take a time to separate or sample a specific blood component layer.

Therefore, an object of the present invention is to provide a sampling device that specifies the position of a predetermined component layer from a sample such as centrifuged whole blood and samples the predetermined component layer.

### Solution to Problem

Aspects of the present invention are exemplified as follows.

### [Aspect 1]

A sampling device sampling a predetermined component layer from a centrifuged sample solution, the sampling device including:
an electrode nozzle;
a nozzle moving mechanism configured to move the electrode nozzle in a vertical direction;
a dispensing tip attached to the electrode nozzle;
a positive electrode and a negative electrode attached to the electrode nozzle and extending along the dispensing tip;
a current detector configured to detect a value of a current flowing between the positive electrode and the negative electrode in the sample solution;
a sample container configured to house the sample solution; and
a controller configured to control the nozzle moving mechanism,
in which the controller drives the nozzle moving mechanism to move the electrode nozzle in the vertical direction, specifies a position of the predetermined component layer from variation of the value of the current, and drives the electrode nozzle to sample the predetermined component layer.

### [Aspect 2]

The sampling device according to aspect 1,
in which a lower end part of each of the positive electrode and the negative electrode includes an exposed conductive surface, and is disposed on a side of a lower end part of the dispensing tip inside the sample container.

### [Aspect 3]

The sampling device according to aspect 2,
in which the lower end part of each of the positive electrode and the negative electrode is disposed at a substantially same height as (for example, within a range of ±1 mm from) the lower end part of the dispensing tip.

### [Aspect 4]

The sampling device according to aspect 1,
in which the sample container is made of a light transmissive material, and the sampling device further includes an optical detector configured to optically detect the component layer inside the sample container.

### [Aspect 5]

The sampling device according to aspect 4,
in which the optical detector includes a photodetector configured to receive light inside the sample container, and an optical detector moving mechanism configured to move the photodetector in the vertical direction.

### [Aspect 6]

The sampling device according to aspect 5,
in which the controller drives the optical detector moving mechanism to move the photodetector in the vertical direction, and specifies the position of the predetermined component layer from variation of light intensity or absorbance detected by the photodetector.

### [Aspect 7]

The sampling device according to aspect 4,
in which the optical detector includes an illuminator configured to illuminate an inside of the sample container.

### [Aspect 8]

The sampling device according to aspect 1, including:
a sample container rack configured to house the sample container; and a
sample container rack moving mechanism configured to move the sample container rack in a horizontal direction.

### [Aspect 9]

The sampling device according to aspect 8,
in which, when the controller specifies the position of the predetermined component layer, the controller moves the sample container rack moving mechanism in the horizontal direction, and sucks the predetermined component layer from the electrode nozzle.

### [Aspect 10]

The sampling device according to aspect 9,
in which the sample container rack moving mechanism includes a first moving mechanism configured to move the sample container rack in a first horizontal direction, and a second moving mechanism configured to move the sample container rack and the first moving mechanism in a second horizontal direction.

### [Aspect 11]

An extracting device including a plurality of sampling devices each according to aspect 1, and extracting an objective substance contained in a predetermined component layer centrifuged from a sample, the extracting device including:
the plurality of sampling devices;
a plurality of swinging containers configured to house the predetermined component layer for extraction of the objective substance; and
a plurality of cartridges for extraction of nucleic acid from the objective substance.

### Advantageous Effects of Invention

The sampling device according to the present invention can specify the position of a predetermined component layer from a sample such as centrifuged whole blood and samples the predetermined component layer.

### BRIEF DESCRIPTION OF DRAWINGS

FIG. 1 is a vertical cross-sectional view illustrating a sampling device according to an embodiment of the present invention.

FIG. 2 is a schematic diagram illustrating a use state of the sampling device illustrated in FIG. 1.

FIG. 3 is a transverse cross-sectional view of an optical detector in FIG. 2.

FIG. 4 is a block diagram illustrating the sampling device illustrated in FIG. 1.

FIG. 5 is a plan view illustrating a bio-related substance extracting device according to the embodiment of the present invention.

FIG. 6 is a side view illustrating a sample container that houses components of centrifuged whole blood.

### DESCRIPTION OF EMBODIMENTS

An embodiment relating to a sampling device and an extracting device according to the present invention is described with reference to the drawings. Parts common to the drawings are denoted by the same reference numerals, and description thereof is appropriately omitted. In the present embodiment, a case where a predetermined component layer (buffy coat layer) centrifuged from whole blood is sampled is described as an example; however, the present invention is not limited thereto, and is applicable to a case where an optional component layer that can be centrifuged from a bio-related substance solution is sampled.

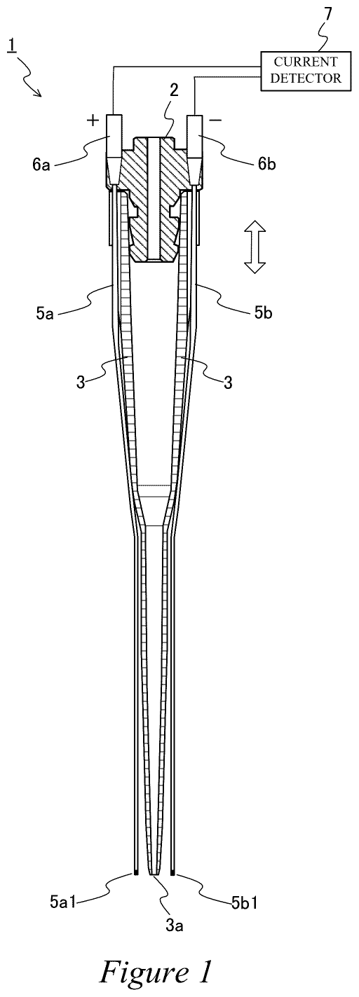

A sampling device 1 according to the embodiment of the present invention is described with reference to FIGS. 1 to 4. As illustrated in FIG. 1, the sampling device 1 includes an electrode nozzle 2 that can suck and discharge a solution, a dispensing tip 3 attached to the electrode nozzle 2, a positive electrode 5a attached to the electrode nozzle 2 and extending along the dispensing tip 3, a negative electrode 5b attached to the electrode nozzle 2 and extending along the dispensing tip 3, a positive terminal portion 6a electrically connected to an upper end part of the positive electrode 5a, a negative terminal portion 6b electrically connected to an upper end part of the negative electrode 5b, and a current detector 7 electrically connected to each of the positive terminal portion 6a and the negative terminal portion 6b through wiring.

Surfaces of the positive electrode 5a and the negative electrode 5b are coated with insulating layers. A positive lower end part 5a1 having a conductive surface exposed without being coated with the insulating layer is provided at a lower end of the positive electrode 5a. A negative lower end part 5b1 having a conductive surface exposed without being coated with the insulating layer is provided at a lower end of the negative electrode 5b. The positive lower end part 5a1 and the negative lower end part 5b1 are disposed on sides of a lower end part 3a of the dispensing tip 3. More preferably, the positive lower end part 5a1 and the negative lower end part 5b1 are disposed at the substantially same height as (for example, within a range of ±1 mm from) the lower end part (opening) 3a of the dispensing tip 3.

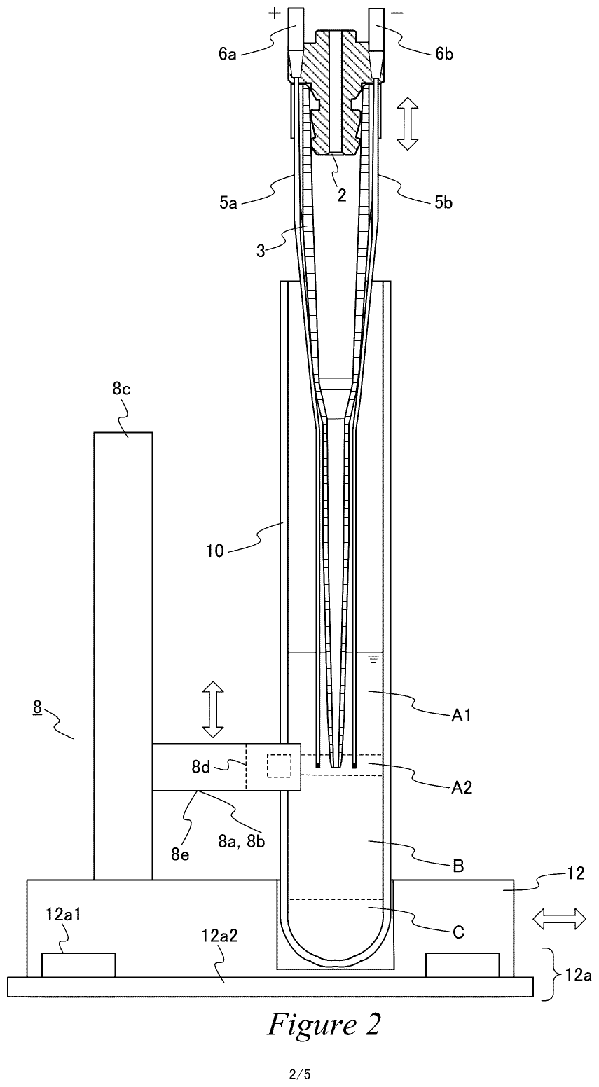

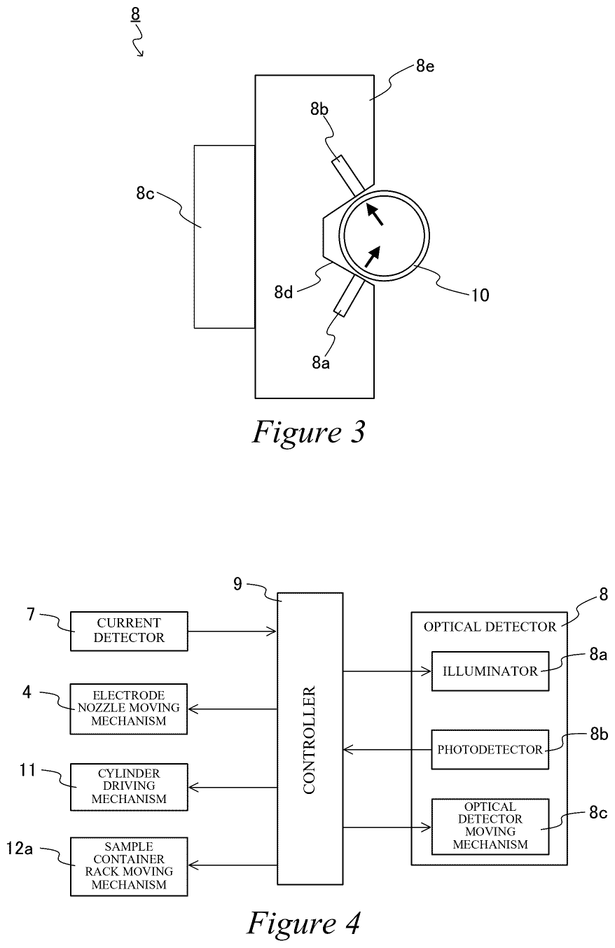

As illustrated in FIGS. 2 and 3, the sampling device 1 includes an optical detector 8 disposed on an outside surface of a sample container 10. The sample container 10 is made of a light transmissive material (such as plastic and glass). The optical detector 8 includes an illuminator (light emitting element such as LED) 8a emitting light toward the sample container 10, a photodetector (photodetector such as photodiode) 8b receiving light having been emitted from the illuminator 8a and passed through the sample container 10, a concave portion 8d receiving a part of the sample container 10, an optical detector housing 8e housing the illuminator 8a and the photodetector 8b, and an optical detector moving mechanism 8c moving the optical detector housing 8e in a vertical direction along the sample container 10. As the optical detector moving mechanism 8c, for example, a rack and pinion, and a driving motor thereof are usable.

As illustrated in FIG. 3, an illumination surface of the illuminator 8a and a photodetection surface of the photodetector 8b are provided in the concave portion 8d, which makes it possible to reduce influence of disturbance light. Further, an illumination direction of the illuminator 8a and a photodetection direction of the photodetector 8b are arranged so as to intersect each other, which makes it possible to avoid relatively strong light emitted from the illuminator 8a from directly entering the photodetector 8b. In place of the illuminator 8a and the photodetector 8b, an imaging element such as a CCD may be provided to specify a position of a predetermined component layer based on difference in color of component layers.

As illustrated in FIG. 2, the sampling device 1 includes a sample container rack 12 housing the sample container 10, and a sample container rack moving mechanism 12a moving the sample container rack 12 in a horizontal direction. The sample container rack moving mechanism 12a preferably includes a first moving mechanism 12a1 moving the sample container rack 12 in a first horizontal direction (x direction), and a second moving mechanism 12a2 moving the sample container rack 12 and the first moving mechanism 12a1 in a second horizontal direction (y direction). Each of the first moving mechanism and the second moving mechanism may include, for example, a belt and a driving motor of the belt.

FIG. 4 is a block diagram of the sampling device 1. A controller 9 of the sampling device 1 is connected to the current detector 7, an electrode nozzle moving mechanism 4, a cylinder driving mechanism 11 driving a cylinder block of the electrode nozzle 2, the sample container rack moving mechanism 12a, the illuminator 8a, the photodetector 8b, and the optical detector moving mechanism 8c. The controller 9 receives a value of a current flowing through a solution inside the sample container 10, detected by the current detector 7. The controller 9 controls operation of each of the electrode nozzle moving mechanism 4, the cylinder driving mechanism 11, and the sample container rack moving mechanism 12a. The controller 9 controls the illuminator 8a of the optical detector 8 to emit light, receives a photodetection signal from the photodetector 8b, and controls operation of the optical detector moving mechanism 8c.

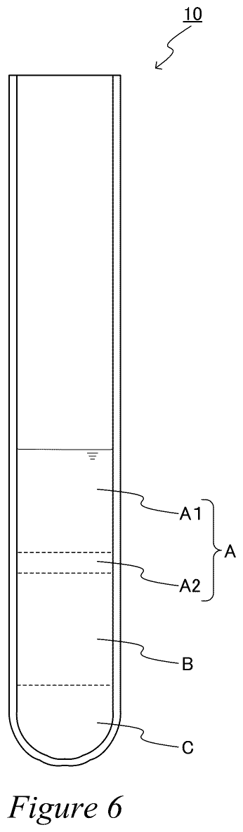

Sampling operation by the sampling device 1 according to the present embodiment is described. First, as illustrated in FIG. 6, whole blood housed in the sample container 10 is separated into component layers A1 to C by using a centrifuge. Thereafter, in a state where the sample container 10 including the component layers A1 to C is housed in the sample container rack 12 as illustrated in FIG. 2, the controller 9 drives the electrode nozzle moving mechanism 4 to lower the electrode nozzle 2 attached with the dispensing tip 3, into the sample container 10 at a constant speed. During the lowering operation, a voltage is applied between the positive lower end part 5a1 and the negative lower end part 5b1, and values of currents flowing through the respective layers are detected by the current detector 7. The current values are varied among the layers due to difference in layer components. Therefore, the controller 9 can detect interfaces of the layers from variation of the current values output from the current detector 7.

Detection of the interfaces by the optical detector 8 is described. The component layers in the sample container 10 are different in refractive index due to difference in components. When the photodetector 8b performs measurement while the optical detector 8 is moved along the sample container 10, a photodetection quantity of the photodetector 8b is varied due to difference in the component layers when the optical detector 8 passes through the interface of each of the layers. By detecting the variation, the controller 9 can detect a position of the predetermined component layer (buffy coat layer A2). Variation of light intensity or a photodetection quantity when the optical detector 8 passes through an interface between a blood serum/blood plasma layer A1 and the buffy coat layer A2 and variation of light intensity or a photodetection quantity when the optical detector 8 passes through an interface between the buffy coat layer A2 and a centrifugal agent layer B are detected by the photodetector 8B. This enables the controller 9 to specify the position of the buffy coat layer in the vertical direction.

When lowering the electrode nozzle 2, the controller 9 also lowers the illuminator 8a and the photodetector 8b at the constant speed same as the electrode nozzle 2. During the lowering operation, the illuminator 8a illuminates each of the layers inside the sample container 10 from the side surface, and the photodetector 8b receives the light having passed through each of the layers from the side surface. The illumination direction and the photodetection direction are preferably arranged so as to intersect each other. Light intensity variation or absorbance variation is detected by the photodetector 8b, which makes it possible to specify the position of the predetermined component layer in the vertical direction.

After the controller 9 specifies the position of the predetermined component layer in the vertical direction by using the current detector 7 and/or the optical detector 8, the controller 9 fixes the lower end part 3a of the dispensing tip 3 in the vertical direction such that the lower end part 3a is positioned inside the predetermined component layer. In the fixed state, the predetermined component layer (buffy coat layer) can be sucked into the dispensing tip 3 while the sample container rack moving mechanism 12 is driven to move the lower end part 3a of the dispensing tip 3 in a lateral direction (x and y directions) in a circle of a transverse cross-section of the sample container 10. The predetermined component layer sucked and sampled into the dispensing tip 3 is discharged into a component layer container by movement of the electrode nozzle 2.

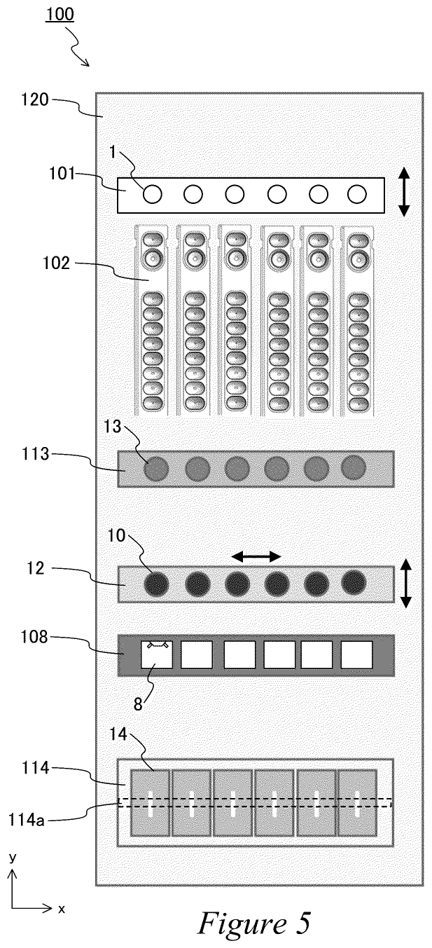

A bio-related substance extracting device 100 including a plurality of sampling devices 1 according to the embodiment of the present invention is described. FIG. 5 illustrates the extracting device 100 that can sample and extract components from a plurality of whole blood samples in parallel. The extracting device 100 includes a horizontal stage 120 on which parts are arranged, and a nozzle unit 101 movable on the stage 120 in the y direction. The nozzle unit 101 includes the plurality of sampling devices 1 (electrode nozzles 2 and electrodes 5a and 5b) arranged in one line along the x direction. The nozzle unit 101 includes the electrode nozzle moving mechanism 4 moving the whole of the nozzle unit 101 in the y direction and a z direction. The extracting device 100 may include a plurality of dispensing nozzles without electrodes.

The stage 120 is provided with a plurality of cartridges 102, an objective substance container rack 113 housing a plurality of objective substance containers 13, the sample container rack 12 housing a plurality of sample containers 10, an optical detector 108 including a plurality of optical detectors 8, and a swinging unit 104 including a plurality of swinging containers 14.

The plurality of cartridges 102 are arranged in one line in the x direction. Each of the plurality of cartridges 102 includes a plurality of wells arranged in the y direction. Each of the plurality of cartridges 102 is used to perform various kinds of treatment such as extraction and purification of an objective substance from the sampled component layer by using the dispensing nozzles and magnetic particles.

The plurality of objective substance containers 13 are arranged in one line in the x direction in the objective substance container rack 113. Each of the plurality of objective substance containers 13 houses the objective substance separated by the swinging container 14.

The sample container rack 12 is movable in the x direction and the y direction by the sample container rack moving mechanism 12a. The optical detector 108 is disposed near the sample container rack 12. When the optical detector 108 is used, the sample container rack 12 is moved in the y direction by the sample container rack moving mechanism 12a, and each of the optical detectors 8 of the optical detector 108 are disposed at positions for measuring the respective sample containers 10 housed in the sample container rack 12.

The swinging unit 114 includes a swing shaft 114a extending in the x direction and a shaft driving mechanism (belt and motor) for integrally swinging the plurality of swinging containers 14 arranged in one line in the x direction. The swinging unit 114 swings the swinging containers 14 under gentler conditions as compared with conditions during suction/discharge using the electrode nozzles 2.

The extracting device 100 can sample a plurality of predetermined component layers from the plurality of sample containers 10 by using the plurality of sampling devices 1 in parallel. The plurality of sampled predetermined component layers are moved to the plurality of swinging containers 14. The plurality of swinging containers 14 are swung under the gentler conditions as compared with the conditions during suction/discharge by the electrode nozzles 2 or the dispensing nozzles, which makes it possible to separate a plurality of objective substances (such as specific cancer cells) bound to magnetic beads with lectin from the plurality of predetermined component layers by using magnets. The plurality of objective substances are moved to the plurality of objective substance containers 13 by the plurality of electrode nozzles 2 or dispensing nozzles. The plurality of objective substances are further moved from the plurality of objective substance containers 13 to the plurality of cartridges 102. Purification, extraction of nucleic acid, and the like using the dispensing nozzles, magnetic particles, and external magnets are performed on the plurality of objective substances on the plurality of cartridges 102.

### REFERENCE SIGNS LIST

1 sampling device
2 electrode nozzle
3 dispensing tip
4 nozzle moving mechanism
5a positive electrode
5b negative electrode
7 current detection unit
8 optical detector
10 sample container
100 extracting device

1. A sampling device sampling a predetermined component layer from a centrifuged sample solution, the sampling device comprising:
an electrode nozzle;
a nozzle moving mechanism configured to move the electrode nozzle in a vertical direction;
a dispensing tip attached to the electrode nozzle;
a positive electrode and a negative electrode attached to the electrode nozzle and extending along the dispensing tip;
a current detector configured to detect a value of a current flowing between the positive electrode and the negative electrode in the sample solution;
a sample container configured to house the sample solution; and
a controller configured to control the nozzle moving mechanism,
wherein the controller drives the nozzle moving mechanism to move the electrode nozzle in the vertical direction, specifies a position of the predetermined component layer from variation of the value of the current, and drives the electrode nozzle to sample the predetermined component layer.

2. The sampling device according to claim 1,
wherein a lower end part of each of the positive electrode and the negative electrode includes an exposed conductive surface, and is disposed on a side of a lower end part of the dispensing tip inside the sample container.

3. The sampling device according to claim 2,
wherein the lower end part of each of the positive electrode and the negative electrode is disposed at a substantially same height as (for example, within a range of ±1 mm from) the lower end part of the dispensing tip.

4. The sampling device according to claim 1,
wherein the sample container is made of a light transmissive material, and
the sampling device further comprises an optical detector configured to optically detect the component layer inside the sample container.

5. The sampling device according to claim 4,
wherein the optical detector includes a photodetector configured to receive light inside the sample container, and an optical detector moving mechanism configured to move the photodetector in the vertical direction.

6. The sampling device according to claim 5,
wherein the controller drives the optical detector moving mechanism to move the photodetector in the vertical direction, and specifies the position of the predetermined component layer from variation of light intensity or absorbance detected by the photodetector.

7. The sampling device according to claim 4,
wherein the optical detector includes an illuminator configured to illuminate an inside of the sample container.

8. The sampling device according to claim 1, comprising:
a sample container rack configured to house the sample container; and a sample container rack moving mechanism configured to move the sample container rack in a horizontal direction.

9. The sampling device according to claim 8,
wherein, when the controller specifies the position of the predetermined component layer, the controller moves the sample container rack moving mechanism in the horizontal direction, and sucks the predetermined component layer from the electrode nozzle.

10. The sampling device according to claim 9,
wherein the sample container rack moving mechanism includes a first moving mechanism configured to move the sample container rack in a first horizontal direction, and a second moving mechanism configured to move the sample container rack and the first moving mechanism in a second horizontal direction.

11. An extracting device including a plurality of sampling devices each according to claim 1, and extracting an objective substance contained in a predetermined component layer centrifuged from a sample, the extracting device comprising:
the plurality of sampling devices;
a plurality of swinging containers configured to house the predetermined component layer for extraction of the objective substance; and
a plurality of cartridges for extraction of nucleic acid from the objective substance.
